(19) United States
(12) Patent Application Publication
(10) Pub. No.: US 2026/0041899 A1
(43) Pub. Date: Feb. 12, 2026

(54) MONITORING DRUG DELIVERY

(71) Applicant: OxSonics Limited, Oxford (GB)

(72) Inventors: Christian Coviello, Oxford (GB); Calum Crake, Oxford (GB); Paul Boulos, Oxford (GB)

(21) Appl. No.: 19/150,061

(22) PCT Filed: Feb. 9, 2024
(86) PCT No.: PCT/GB2024/050358

§ 371 Date: Jul. 22, 2025

(30) Foreign Application Priority Data
Feb. 10, 2023 (GB) 2301928.4

(51) Int. Cl. A61M 37/00

(52) U.S. Cl. CPC A61M 37/0092; A61M 2205/3375; A61M 2205/702

(57) ABSTRACT

Apparatus for monitoring drug delivery to a subject comprises: transmission means (201, 202) for transmitting pressure waves towards the subject thereby to generate cavitation at the subject: an array of detectors (202) for detecting pressure waves generated by the cavitation; and processing means (210) arranged to receive signals from the detectors (202), and to process the signals to determine the location (214) of the cavitation and at least one further parameter of the cavitation; and from the further parameter to generate an efficacy indicator indicative of efficacy of drug delivery at the location of the cavitation.

### FIELD OF THE INVENTION

The present invention relates to therapeutic ultrasound systems and in particular to the mapping, monitoring and control of treatments using acoustic (ultrasound-induced) cavitation.

### BACKGROUND TO THE INVENTION

The use of focused ultrasound (FUS) for cancer therapy has several major advantages over other, more established treatment modalities: it is cheap, non-invasive, and has minimal side-effects. However, widespread acceptance of FUS is hindered by the lack of a reliable real-time monitoring system.

Above a certain pressure threshold, high-amplitude acoustic waves propagating through tissue can spontaneously nucleate and excite small, nano- or micron-sized bubbles, a phenomenon known as acoustic cavitation. With the addition or infusion of exogenous bubbles acting as cavitation nuclei, be they shelled bubbles or stabilized bubbles on the surface of solid particles [Stride, E. P., & Coussios, C. C. (2010). Cavitation and contrast: The use of bubbles in ultrasound imaging and therapy. Proceedings of the Institution of Mechanical Engineers, Part H: Journal of Engineering in Medicine, 224 (2), 171-191], lower amplitude acoustic waves can cause acoustic cavitation in the nuclei, enabling a lower pressure threshold. Depending on the desired application, the cavitating bubbles can be used to cause desired bioeffects. In the case of tissue ablation, these bioeffects could cause thermal necrosis or mechanical fractionation of tissue. In drug delivery, this could be cavitation-induced micro-streaming or micro-pumping, permeation of vasculature, and poration of cells, which all aid or enhance the transport of unmodified drugs across tissue membranes or alternatively directly act to rupture drug-loaded vehicles for targeted drug delivery.

The bubbles can also re-emit part of the incident ultrasound over a range of frequencies that are different to the FUS excitation frequency. These emissions have a higher frequency content than the original FUS source, and will be absorbed more readily by surrounding tissue, enhancing heat deposition in thermal ablation. While the field of FUS broadly covers all biomedical applications and system configurations involved with focusing ultrasound, the more specific term high-intensity focused ultrasound (HIFU) is generally used when the amplitude or intensity of incident ultrasound is higher than achievable from a diagnostic ultrasound imaging system. However, the general term “focused ultrasound” here refers to applications of focusing ultrasound for therapeutic purposes, whether it is HIFU or utilizes lower intensity ultrasound.

WO20100052494 discloses a system for passive acoustic mapping (PAM) which images a region of acoustic cavitation, by passive detection of ultrasound generated by the cavitation, during FUS treatment. WO2021/255433 describes methods of mapping cavitation activity in three dimensions and registering a map of cavitation with a 3D image of an organ or tissue so that the position of the cavitation on the organ or tissue can be monitored in real time. WO2011/036485 describes the use of a cavitation detection system to monitor parameters of treated tissue.

In radiation therapy various methods are known for monitoring and controlling radiation dose in the tissue to ensure that the treatment is sufficient but not excessive. However radiation therapy of cancerous cells works directly on cells to break the DNA chains that control cell division. A dose required for a particular cell type's radio sensitivity can be empirically measured, modeled, and in-situ monitored during therapy. Thus, dosimetry in radiation therapy is based solely on the dwell time of the radiation beam to achieve a certain total fluence dose based on beam size and number of angles for a particular tumor. Pre-treatment planning from pre-collected imaging and mathematical modeling (and system calibration using gel phantoms) is used in conjunction with real-time in-situ dose measurements from monitor units to determine if the correct dose has been delivered and treatment is complete. Thus, multiple direct measurements of the phenomena that causes cell death are used, together with calibration and modeling of prior measurements of cell death from radiation exposure.

### SUMMARY OF INVENTION

PAM dosimetry cannot measure the direct phenomena that cause improved delivery (micro streaming or drug pumping of a drug) that then leads to improved efficacy (of drug delivery). Instead the system can only measure a surrogate (e.g. the amount of cavitation energy) and not the amount of actual micro-pumping that is occurring. The local blood concentration of cavitation nuclei will vary, and certain tumor types will have vastly different distributions of blood vessels. Further, limitations of the acoustic field-of-view to the array on measuring bubbles, bubble shielding, and diffraction/reflection from tissue boundaries, bones, and other physiological structures will also limit the acoustic energy received. Additionally, the inherent randomness of cavitation nucleation means that even for an increasing amount of ultrasound energy (above the cavitation threshold pressure) transmitted, the detected cavitation energy does not increase in a simple linear manner outside a limited range. Therefore, the relatively simple methods of radiation therapy dosimetry are not applicable to cavitation-assisted drug delivery. However in embodiments of the present invention in-vivo models (usually animal models with xenografted or allografted tumors) can be used to get a base understanding of the amount of drug that can be delivered in a certain tumor model, and then based on a measured “vascularity index” or known degree of vascularity of that model, extrapolations can be made for other models. This then allows mathematical modeling of dose required for a particular patient based on their disease burden, with PAM providing the in-situ estimate of “cavitation dose” that leads to a measure of sufficiency of treatment.

The present invention therefore provides apparatus for monitoring drug delivery to a subject, the apparatus comprising: transmission means for transmitting pressure waves towards the subject thereby to generate cavitation at the subject; an array of detectors for detecting pressure waves generated by the cavitation; and processing means arranged to receive signals from the detectors, and to process the signals to determine the location of the cavitation and at least one further parameter of the cavitation; and from the further parameter to generate an efficacy indicator indicative of efficacy of drug delivery at the location of the measured cavitation.

The processing means may comprise a memory having stored therein a relationship between values of the measured parameter and values of the efficacy indicator. The processing means may be arranged to generate the efficacy indicator from the measured parameter and the stored relationship. The stored relationship may comprise a lookup table.

The processing means may further be arranged to map the location of the cavitation onto an image of the subject.

The image of the subject may include a definition of a region for treatment.

The transmission means may be arranged to transmit the pressure waves towards a focal region. The processing means may be arranged to move the focal region whereby cavitation is generated at a plurality of locations within the subject.

The transmission means may be arranged to transmit the pressure waves over a plurality of transmission periods, and to move the focal region between the transmission periods.

The transmission means may be manually movable to move the focal region within the subject.

The efficacy indicator may comprise a control signal. The processing means may be arranged to control display means to display an image of the subject. The efficacy indicator may further comprise an efficacy image feature displayed on the display means. The control signal may be arranged to control the display means to display the efficacy image feature together with the image of the subject.

The efficacy image feature may be located on the image of the subject at a position corresponding to the position of the cavitation in the subject.

The processing means may be arranged to generate an efficacy indicator for each of a plurality of regions of cavitation in the subject. Alternatively, or in addition, the processing means may be arranged to generate an efficacy indicator from all of the regions of cavitation.

The efficacy indicator may comprise a control signal. For example the control signal may be arranged to control the display means to display an efficacy image feature. Alternatively, or additionally, the control signal may be arranged to control an interface to provide information to the clinical user, for example via the display means, an audio interface or a tactile interface, providing instructions on how to manipulate the ultrasound probe to control the location of cavitation, for example in the case of a hand-held probe. Alternatively the control signal may be arranged to control the transmission means. For example it may be arranged to start or stop the transmission of the pressure waves, for example to trigger a pulse of pressure waves. The control signal may be arranged to control the location of the cavitation. For example, it may be a control input to a robot actuator, or a control input to a beam steering system of the transmission means.

The at least one further parameter of the cavitation may comprise at least one of detected ultrasound energy, mean detected ultrasound energy, maximum detected ultrasound energy, thresholded detected ultrasound energy, or other filtered ultrasound energy as a combination of these metrics.

The detected ultrasound energy may be the ultrasound energy measured as originating from a particular position in the subject. It may be an image element (pixel or voxel) value of an ultrasound image, in particular a PAM image. It may be the energy detected over a detection period.

The processing means may be arranged to identify a plurality of cavitation regions. The at least one further parameter may comprise the relative positions of the cavitation regions. The processing means may be arranged to process the signals received over a plurality of detection periods, and to identify one of said cavitation regions associated with each of the detection periods.

The apparatus may further comprise a user input for inputting a tissue type and/or a drug class, wherein the efficacy indicator is dependent on the tissue type and/or the drug class. The drug class may comprise a group of drugs or a single drug.

The system may further comprise, in any workable combination, any one or more features of the embodiments of the invention, shown in the accompanying drawings, as will now be described.

### BRIEF DESCRIPTION OF THE DRAWINGS

FIG. 1 is a schematic diagram of an ultrasound system according to an embodiment of the invention;

FIG. 2 is a schematic diagram of the system of FIG. 1 transmitting ultrasound waves for anatomical imaging;

FIG. 3 is a schematic diagram of the system of FIG. 1 transmitting ultrasound waves for therapeutic treatment;

FIG. 4 is a flow chart showing how an efficacy measure may be obtained from measured parameters;

FIG. 5 is a flow chart showing how an efficacy measure may be obtained from measured parameters;

FIG. 6a shows two images of cavitation in the system of FIG. 1 with one, and a plurality of, indicators of efficacy;

FIG. 6b is a 3D image of a target treatment region with one, and a plurality of, indicators of efficacy;

FIG. 7 shows different distributions of indicators of efficacy on an image of a target region;

FIG. 8 shows build-up of indicators of efficacy on an image of a target region during pulsed treatment;

FIG. 9 shows build-up of indicators of efficacy on an image of a target region during continuous treatment;

FIG. 10 shows different indicators of efficacy on an image of a target region during continuous treatment;

FIG. 11 is a schematic diagram of an ultrasound system according to a further embodiment of the invention;

FIG. 12 is a schematic diagram of an ultrasound system according to a further embodiment of the invention;

FIG. 13 is a schematic diagram of an ultrasound system according to a further embodiment of the invention;

FIG. 14 is a plot showing the relationship between treatment efficacy and average ultrasound energy for experimental examples;

FIG. 15 is a plot showing the relationship between treatment efficacy and maximum ultrasound energy for experimental examples;

FIG. 16 is a plot showing the relationship between treatment efficacy and cumulative ultrasound energy for experimental examples;

FIG. 17 is a 2D image of cavitation in a subject;

FIG. 18 is a plot of maximum ultrasound energy over time in the subject of FIG. 17;

FIG. 19 is a 2D image of cavitation in a different subject;

FIG. 20 is a plot of maximum ultrasound energy over time in the subject of FIG. 19;

FIG. 21 is a plot of tumour volume over time in a number of subjects;

FIG. 22 is a plot of maximum ultrasound energy and cumulative ultrasound energy in the subjects of FIG. 21;

FIG. 23 is a plot of maximum ultrasound energy and mean ultrasound energy in the subjects of FIG. 21;

FIG. 24 is a plot of maximum ultrasound energy over time for one of the subjects of FIG. 21;

FIG. 25 is a plot of maximum ultrasound energy over time for one of the subjects of FIG. 21;

FIG. 26 is a plot of maximum ultrasound energy over time for one of the subjects of FIG. 21;

FIG. 27 is a plot of maximum ultrasound energy over time for one of the subjects of FIG. 21;

FIG. 28 is a plot of thresholded ultrasound characteristics in the subjects of FIG. 21;

FIG. 29 is a plot of thresholded ultrasound energy in one of the subjects of FIG. 21;

FIG. 30 is a plot of thresholded ultrasound energy in one of the subjects of FIG. 21;

FIG. 31 is a PAM image of cavitation within a subject;

FIG. 32 is a plot of maximum cavitation energy within the subject of FIG. 31;

FIG. 33 is a PAM image of cavitation within a subject;

FIG. 34 is a plot of maximum cavitation energy within the subject of FIG. 33;

FIG. 35 is a PAM image of cavitation within a subject;

FIG. 36 is a plot of maximum cavitation energy within the subject of FIG. 35;

FIG. 37 is a PAM image of cavitation within a subject;

FIG. 38 is a plot of maximum cavitation energy within the subject of FIG. 37.

### DESCRIPTION OF THE PREFERRED EMBODIMENTS

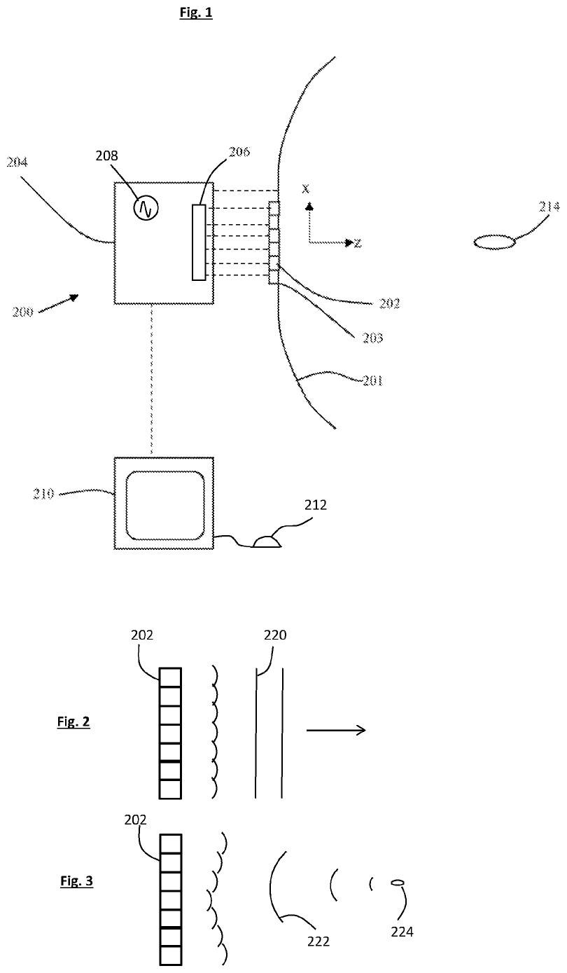

Referring to FIG. 1, the ultrasound system 200 may comprise a geometrically focused ultrasound transducer 201, with an array of ultrasound transducer elements 202 positioned in an aperture 203 in the centre of the transducer 201. Each of the transducer elements 202 may be operable to generate ultrasound and also to detect ultrasound. They may therefore be usable in an active mode in which they generate and detect ultrasound to generate reflective (e.g. B-mode) ultrasound images, or in a passive mode in which they only detect ultrasound. The array may be a linear or convex linear array extending primarily in a direction which will be referred to as the x direction as shown in FIG. 2. The direction, perpendicular to the x direction, along the axis of the transducer will be referred to as the z direction. The imaging plane of the array is therefore the x-z plane. The direction perpendicular to both the x and z directions will be referred to as the y direction.

A control unit 204 is arranged to control generation of ultrasound signals by each of the transducer elements 202 and to receive the detection signals from each of the transducer elements 202. The control unit 204 may comprise a pre-amplifier and filter block 206 and an oscillator 208. The control unit 204 is also arranged to control the transducer 201 to control the power and frequency of the ultrasound generated by the transducer 201, for example using a signal from the oscillator 208 to control the frequency of the ultrasound. It will be appreciated that the control unit 204, while being described functionally, can be made up of a single processor, or two or more separate processors performing different functions, for example control and analyzing functions, within the system. The control unit is connected to a display screen 210 on which data derived from the detection signals can be displayed in a suitable format, typically an image of part of a patient including a target region for treatment such as a tumor, an image of cavitation, and any indicators for showing a clinician the efficacy of the treatment. A user interface, which may be a graphical user interface (GUI) such as a mouse 212, trackball, or touchpad, operating with the display screen 210, or a touchscreen, is provided to enable a clinician to interact with an image on the display 210 thereby to modify the image displayed and provide control inputs to the control unit 204 to control the treatment and imaging performed by the system. The user interface may also include an audio interface provided by one or more speakers which can also provide information or instructions to the user, or a tactile interface arranged to provide tactile information or instructions to the user. In this case, the therapeutic transducer 201 has a focus in a focal region 214, in which it will generate the highest intensity ultrasound.

While the arrangement of FIG. 1 can be implemented using a variety of components and systems, in one embodiment an ultrasound data acquisition system may be used that allows simultaneous raw radio-frequency (RF) data or in-phase quadrature (I/Q) data-which can be demodulated to RF-across a wide ultrasound bandwidth (e.g. 1-15 MHz) from multiple individual elements 202. If the array is to be used in the passive mode, pulse transmission may be switched off so that the array acts on receive only. In some modes, one group of transducer elements 202 is used in the active mode and another group in the passive mode so that active and passive detection can be used simultaneously. To make the system clinically applicable, a therapeutic ultrasound transducer 201 may be used, which has a central aperture 203 for a linear (straight or convex) detector array 202.

The theory behind the operation of the passive acoustic mapping will now be briefly described, although it is described in more detail in WO2010/052494. Active detection, which includes pulse-echo (e.g. B-mode) imaging, requires an ultrasound generator which is arranged to generate ultrasound, typically in a pulse, and a detector, or receiver, which detects reflected ultrasound or re-emitted ultrasound from a cavitation region, and a processing system which uses the time interval between the generation of the ultrasound and the detection of ultrasound in determining the position of detected cavitation. In contrast, in passive localization and mapping, there is no direct information about the propagation time from a source to a receiver. Instead, cross-correlation of detection signals from a pair of receivers can provide an estimate of the differential time of arrival (DTOA), i.e. the difference in arrival time at the receivers of a signal from a source. This enables the difference in distance between the receivers and the source to be estimated. By using a set of cross-correlation pairs, single source localization and extended source mapping is possible. It will be appreciated from this that a single detector can be operated in both active and passive detection, depending on the processing which is performed on the sensor signals. A further explanation of passive localization and imaging follows.

Typically, where there is an extended region of cavitation, spatial maps of cavitation are necessary. The approach typically taken is one of passive beamforming, and one method is described in Coviello, et al. “Passive acoustic mapping utilizing optimal beamforming in ultrasound therapy monitoring”, J. Acoust. Soc. Am. 137 (5), May 2015. If a regular grid of pixels is defined in the imaging plane in which to create the spatial maps, each pixel is defined by a center defined by a 3D coordinate x=[x, y, z]^{T}, and a spacing in each dimension given by [dx, dy, dz]^{T}. Define the distance from pixel position x to receiver j at position xj by

$${d}_{j} ( x ) = \sqrt{{( x - {x}_{j} )}^{2} + {( y - {y}_{j} )}^{2} + {( z - {z}_{j} )}^{2}} .$$

For each pixel, the data at the receiver is pre-steered with time delays applied to each receiver signal to compensate for the propagation time from the pixel being imaged to the receiver. If we assume there are N total receivers, the strength of the sound field at the pixel position is then

$$q ( x , t ) = \frac{4 π}{N α} {\sum }_{j = 1}^{N} {d}_{j} ( x ) {s}_{j} ( t + {τ}_{j} ( x ) )$$

Where sⱼ(t) is the recorded signal at receiver (sensor) j, τⱼ(x)=dⱼ(x)/c the propagation time from location x to sensor j, a is the piezoelectric coefficient that is a characteristic of each receiver (sensor) element, and c is the speed of propagation. The multiplication by dⱼ(x) above compensates for the reduction in source signal strength from spherical spreading of the wavefronts. An image of source energy at a pixel is generated by integrating the square of the source strength over a collection period of length T, assuming uniform detector weighting as

$$Ψ ( x ) = \frac{1}{4 π {ρ}_{0} c} {\int }_{{t}_{0}}^{{t}_{0} + τ} {q ( x , t )}^{2} d t$$

where ρ₀ is the density of the medium and c is the speed of propagation. This energy is then computed for each pixel position desired in the image.

Referring to FIGS. 2 and 3, the control unit 204 may be arranged to control the transducer elements 202 as a phased array. For example the control unit may be arranged to generate a transmit signal for each of the transducer elements 202 to control the frequency and timing, i.e. relative phase, of the vibration, and hence the ultrasound, that each transducer element produces. Typically the frequency of vibration of each of the transducer elements is controlled so as to be the same, and the phase, or timing, of each of the elements is varied so as to steer the ultrasound that is generated by the array as a whole. The transducer elements may be arranged to vibrate in phase with each other, which produces ultrasound having straight parallel wave fronts 220 all travelling in the same direction, as shown in FIG. 2. This is suitable for anatomical ultrasound (B-mode or other reflective) imaging. If the phase of the vibrations of the elements 202 is shifted so that those at the outer ends of the array are in phase with each other, and the delay increase towards the centre of the array, as shown in FIG. 3, then this generates ultrasound with curved wave fronts 222 that converge at a focal region 224. The position of the focal region 224 can be varied by varying the relative phase of the vibrations of the transducer elements 202.

It will be appreciated that rather than having a separate transducer 201 for the focused ultrasound (FUS) transmission, the array of ultrasound elements 204 may be used to generate ultrasound for both anatomical imaging and therapeutic FUS by switching rapidly between the two different phase configurations. This allows the transducer arrays to be incorporated in a hand-held probe, as described in WO2021/255433. Furthermore, since both anatomical ultrasound imaging and the passive acoustic mapping described above require detection of ultrasound reflected or generated by the tissue being imaged, this detection may also be done in some embodiments of the invention by the same transducer elements 202 that are used to transmit ultrasound. However that requires further time division and in many cases it is preferable to have separate a transducer array for detection, or receiving, of ultrasound. Therefore, in the following description where reference is made to transmit and receive arrays these will typically be separate arrays, but may alternatively be the same array.

As described in WO2021/255433 the system of FIG. 1 may be arranged to image cavitation resulting from FUS during treatment and to locate the cavitation activity in two or three dimensions by registering a map of the cavitation with a 3D image of an organ or tissue so that the position of the cavitation on the organ or tissue can be monitored in real time.

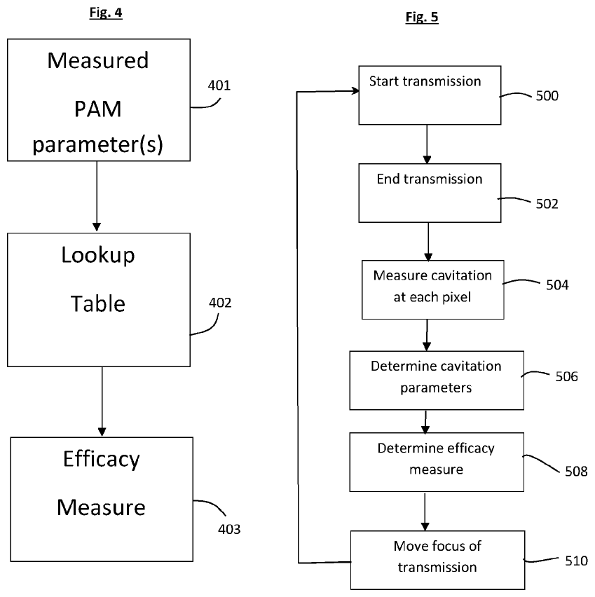

Referring to FIG. 4, during operation, the FUS pulse produces cavitation within a region of the subject, which may be referred to as the cavitation region. The cavitation region will generally be within the focal region 214, but the two may not exactly coincide, because the level of cavitation is also dependent on the nature of the tissue which may vary within and around the focal region, the processor 204 is arranged to monitor cavitation in the cavitation region using the detection signals from the transducer elements 202. From the detection signals, the processor 204 is arranged to derive parameters 401 of the cavitation. From those parameters 401 of cavitation, the processor 204 is arranged to derive one or more efficacy indicators, which may be derived from one or more efficacy measures 403 which provide a measure of the efficacy of the treatment in the cavitation region. Typically the detection signals are sampled over a detection period and the sample values stored in a buffer. The length of the detection period depends on the sample rate, the number of detector elements, and the size of the buffer. The data from the buffer can then be processed to form a PAM image.

Typically the PAM data is collected once for each FUS pulse and is in the form of image data, comprising a value for each of a number of voxels. Each of the voxel values represents the total ultrasound energy, within a frequency band appropriate for PAM, detected as originating from a position in the subject corresponding to the position of the voxel within the image, over a detection period. The detection period may follow the transmission of the FUS pulse. However it is also possible for the collection period to overlap at least partly the transmission period if the system is set up to transmit and receive ultrasound simultaneously. It is also possible for the PAM data to be collected multiple times over a sequence of consecutive detection periods after each FUS pulse. In that case the data can be used to generate multiple PAM image frames after each FUS pulse, or the multiple voxel values for each voxel following each pulse may be summed to form a single ‘averaged’ PAM image. In a system in which simultaneous FUS transmission and PAM detection is possible, the FUS transmission may be continuous rather than pulsed. The PAM data can then be collected in a series of detection periods.

From each PAM image, various parameters of the cavitation region can be determined. The centre of the cavitation region can be identified. This may be by identifying the voxel, or the group of voxels, with the maximum energy value. Alternatively the mean or median position of detected cavitation, or the centroid of the cavitation region may be used. A series of FUS pulses and PAM images may be produced over an insonation period, and the measured cavitation over that insonation period may be defined by a number of cavitation parameters.

Examples of the parameters 401 of cavitation include the level of cavitation, which may be measured as the energy of the ultrasound, within the frequency band of cavitation-generated ultrasound, detected by the transducer elements 202. This energy measurement corresponds to the PAM image voxel values. More specifically this may be measured as a peak energy, for example the highest voxel value over the whole insonation period, or the mean energy over an insonation period for just one voxel or a number of voxels in the cavitation region. Alternatively one of the cavitation parameters may be the period of time over which the energy exceeds a threshold level, for example measured as the number of PAM frames or FUS pulses over the insonation period for which the maximum voxel value exceeds a threshold value. The size of the cavitation region may be determined, for example as the number of voxels having a value above a threshold. The total energy, i.e. the sum of voxel values, over the whole cavitation region may be determined. Another important parameter of the cavitation is its location, which as mentioned above may be determined in a variety of ways. The cavitation parameters used to derive the efficacy indicator may therefore include the position of the cavitation region relative to a target region, which may be fixed or may vary over an insonation period, or the positions of multiple cavitation regions relative to the target region, and/or relative to each other, or the size of the cavitation region or each cavitation region.

The efficacy indicator may indicate whether the cavitation parameter exceeds a threshold in the cavitation region. For example if the cavitation parameter is the peak or mean measured ultrasound energy over an insonation period, then a simple threshold, or a number of different thresholds, of those values may be used to determine whether or not the treatment was effective, or the degree to which it was effective. Alternatively the cavitation parameters may be used to derive an efficacy measure which is then used to determine whether, or to what extent, the treatment was effective, and the indicator derived from that determination. Examples of the efficacy measure 403 include an estimated dose of a drug delivered to the focal region, or a proportion of a required dose of a drug delivered to the focal region.

The derivation of the efficacy indicator may be made using a lookup table 402. The lookup table may for example include a number of tissue categories. If the efficacy indicator is derived directly from the cavitation parameter(s), then the lookup table may provide a value of the efficacy indicator for each value (or range of values) of the cavitation parameter for each tissue type, and for each of a number of drug classes (each class including multiple drugs, or a single drug in the extreme). If an efficacy measure is derived from the cavitation parameter(s), then the lookup table may define a relationship between the measured cavitation parameters and the efficacy measure(s) for each of the tissue categories, and for each of a number of drug classes. Examples of the tissue categories include levels of tissue vascularity, types and/or quantities of extracellular matrix ECM) and degrees of desmoplasticity, as well as the anatomical location of the tissue. For example, it is known to categorize the vascularity of tissue using color Doppler images of the tissue, which effectively measures blood flow in the tissue, and forming a histogram of the color pixels, and normalizing by tumor volume to get an “index” of vascularity. The categorization may be also experimental based on extrapolation from preclinical experiments with xenograft or allograft tumors in small or large animals, or even from prior clinical samples of treated patients in a clinical investigation. This clinical information could be derived from radiological images of perfusion (using contrast CT or MR), avidity or metabolic response (from PET/CT), or tumor size changes from treatment (from CT or MR). The user interface 212 may be used to input or select a tissue type and/or a drug class (or individual drug), and the processor 204 may be arranged to select a value of the efficacy indicator based on the selected tissue type and the selected drug class (or individual drug), as well as the cavitation parameters.

Whilst the system can be set up initially using modelled data or preclinical data to define a suitable lookup table, it will be appreciated that over time, clinical trial data can be used to refine the lookup table so as to improve the accuracy of the efficacy indicators generated, and to increase the number of different tissue types and/or drug classes that can be selected.

Referring to FIG. 5, typically ultrasound is directed at one focal region for a fixed time period or pulse period, for example 1 ms, following which the cavitation in a field of view including the focal region is detected and imaged using PAM. The location of the cavitation region and efficacy of the treatment at that location is determined from the PAM data. Feedback to the clinician regarding each transmission pulse may be provided for example as colored shape, typically a circle, superimposed on an image of the treatment target in the position of the detected region of cavitation. In general terms the method may therefore comprise starting the focused ultrasound transmission at step 500, ending the transmission at step 502, measuring cavitation at each pixel or voxel of the PAM image at step 504, determining the relevant cavitation parameters, at step 506, determining a measure of treatment efficacy, for example using the lookup table, at step 508, and then moving the focal region at step 510 and starting a new transmission. The full process for treatment with efficacy measurement requires planning and preparation, and may include the following steps.

Planning: based on a full patient profile from 3D imaging pre-loaded onto the control unit 204, software in the system completes tissue and tumor/target segmentation through automated and semi-automated image processing. The clinician can select tumors or other targets to be treated and highlight them using the GUI 210.

Registration: using software in the control unit 204 the clinician completes semi-automated registration of the live B-mode ultrasound imaging plane to the labelled and segmented 3D MR/CT image for improved treatment guidance. Based on the pre-selected tumors, target treatment volumes for treatment, which will typically include a tumor and a margin around the tumor, are displayed in 2D and 3D views on the screen 210 to the clinician.

Treatment: infusion of cavitation nuclei, that are optimized for use with the ultrasound system, and the drug prescribed for patient is started. Using the imaging system, typically in the form of a hand-held probe, and the GUI, the clinician aligns a crosshair on screen, the position of which is determined from the position of the probe relative to the target region and the known position of the focal region relative to the probe, with target volume and initiates a focused ultrasound pulse, for example by pressing a treatment trigger, to send focused ultrasound to the target volume.

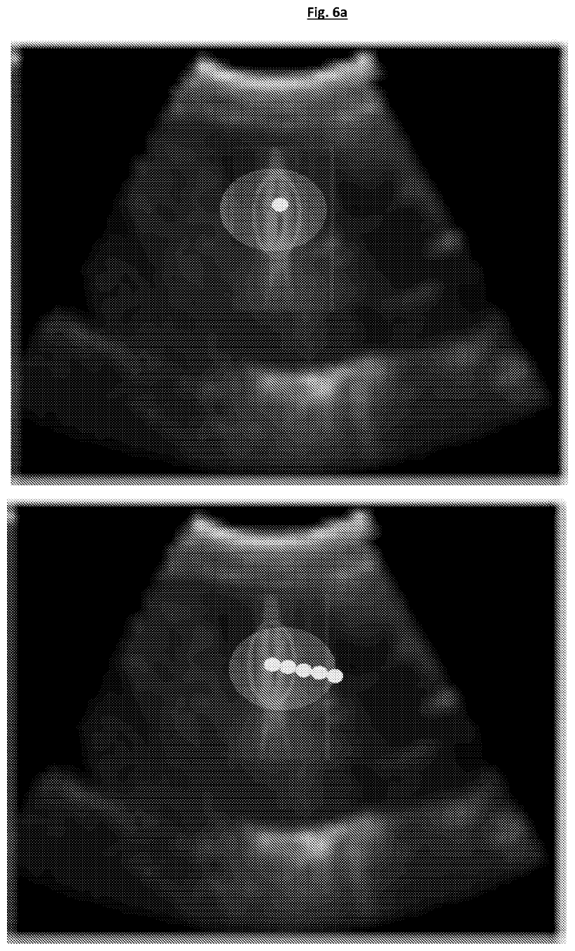

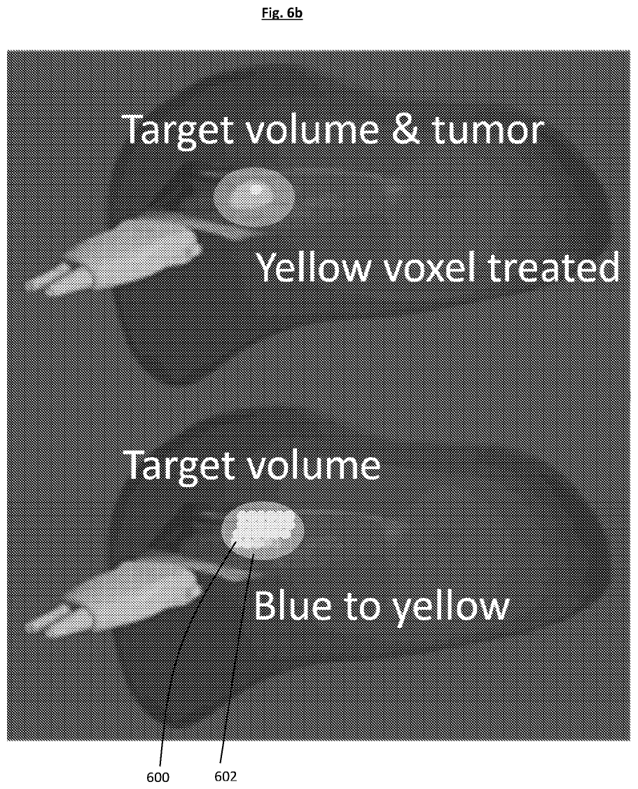

Feedback/Dosimetry: GUI 210 shows both a PAM/B-mode 2D view of live cavitation, as shown in FIG. 6a, and a 3D PAM voxelated view including an efficacy indicator at each position where a focused ultrasound pulse has been delivered and cavitation detected, as shown in FIG. 6b. The efficacy indicator may be provided for each position and may be based, for example, on the amplitude of cavitation or time/pulse duration at each position. For example the target volume 602 in the 3D image may be indicated in one colour, such as blue, and an indicator 600, in the form of an image feature showing the position of detected cavitation from each focused ultrasound pulse, may be superimposed on the target region in a different colour, such as yellow. The clinician can then continue moving the focal region and triggering pulses of f18ltrasoundtrsound until the target region, or a sufficient proportion of it, has been treated, as indicated by its change of colour in the 3D image, for example from blue to yellow. Alternatively the efficacy indicator may be provided for the target as a whole, for example based on spatial coverage (% of target volume). The clinician can raster the focal region of the focused ultrasound through the treatment volume using the feedback based on dosimetry until the treatment is complete, which may be indicated by the system via the GUI, or determined by the clinician based on the efficacy indicators provided by the system.

Documentation: when the treatment is complete, which the GUI may indicate to the clinician, the clinician can end the treatment. Automated reporting documents the treatment coverage, and saves to patient records.

Referring to FIG. 7, in one embodiment the efficacy of the treatment may be indicated by means of a single indicator 700 for each insonation period which may comprise a pulse, or sequence of pulses, of focused ultrasound. The indicator may be a simple circle superimposed on the image of the target volume 702 indicating that a threshold level or duration of cavitation has been generated at the position in the tissue where the cavitation region from the focused ultrasound was located during the insonation period. If one or more focused ultrasound pulses are transmitted into the tissue but the cavitation parameters indicate that that has not caused sufficient cavitation to be effective, then either no indicator is shown at the position of the focal region (or cavitation region if cavitation is detected) for that insonation period, or a different indicator, such as a circle of a different colour, may be shown at that position. After each insonation period the clinician can move the probe to move the focal region and trigger another insonation period, which treats another cavitation region of the tissue, while the system monitors the cavitation at that position, assesses the efficacy of treatment at that position, and generates another indicator 700 at the new position on the image if it is effective. As the treatment continues, a series of indicators is built up on the image of the target volume 702. The overall effectiveness of the treatment will depend on the number of effective insonation periods, and the positions of the different cavitation regions throughout the target volume 702. For example the left hand image of FIG. 7 shows an even distribution of effective treatment positions through the target volume 702, which may provide effective treatment of the tumour, whereas the right hand image shows the same number of effective treatment positions but concentrated in one part of the target volume, which may not provide effective treatment of the tumour. The assessment of the overall treatment may be made by the clinician during treatment, in which case the clinician may continue treating further positions until the whole tumour is successfully treated. Alternatively the control unit 204 may analyse the number and distribution of the successfully treated positions as the treatment progresses and provide a further indication, such as the green tick 704 in FIG. 7, once the target volume has been sufficiently treated.

Referring to FIG. 8, in another embodiment, the overall assessment of the efficacy of the treatment is determined on the basis of the total duration of cavitation anywhere within the target volume 802. In a pulsed focused ultrasound system in which the pulses are transmitted continuously while the clinician moves the probe this may be indicated by the colour of the indicator associated with each focused ultrasound pulse for which cavitation, or sufficient cavitation, is detected. The cavitation detected for the first pulse is indicated by an indicator 800 of a first colour. The cavitation detected for each subsequent effective pulse is indicated by an indicator of the same or a different colour. Finally when cavitation from a sufficient number of effective pulses has been detected within the target volume, the indicator 806 for the last pulse is displayed in a different colour to indicate to the clinician that treatment is complete.

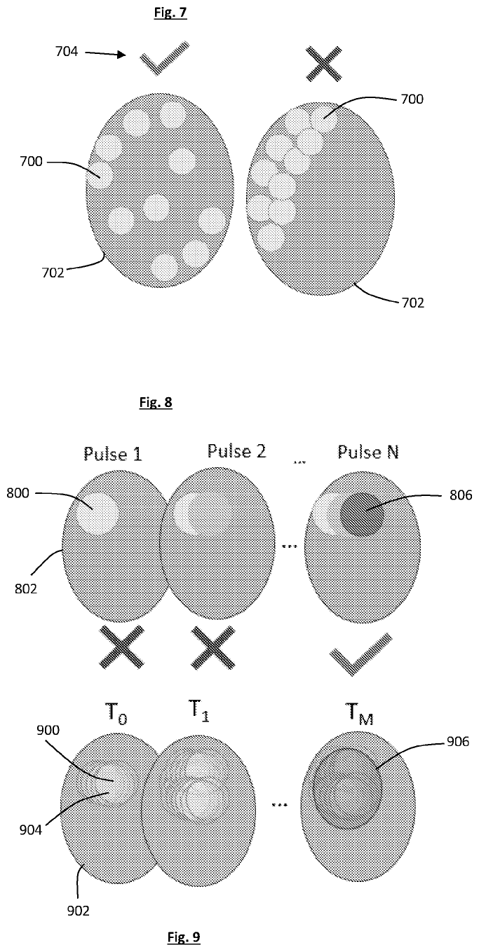

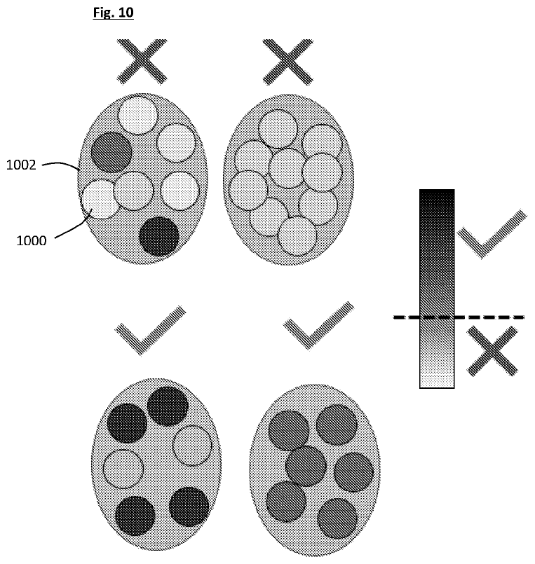

Referring to FIG. 9, in a continuous focused ultrasound system, the location 900 of the detected cavitation within the target volume 902 may be continuously monitored and displayed, for example changing the colour of the display in any region 904 where the cavitation region where cavitation has been generated. The treatment may be deemed effective either after cavitation has been generated within the target volume for a predetermined length of time, or after the total volume 906 within which cavitation has been generated reaches a predetermined size. This assessment may be made by the clinician on the basis of the visual feedback as shown in FIG. 9, or it may be made by the control unit which may be arranged to calculate the total volume 906 repeatedly during treatment and determine when it reaches a predetermined value, which may for example be an absolute volume, or a proportion of the target volume 902. This determination may result in the control unit generating a further visual indicator to the user on the display, or an audible indicator to the user via a speaker, or a tactile indicator, for example to provide information or directions to the user, or generating a control signal for example to stop the treatment.

Referring to FIG. 10, in a further embodiment with a pulsed focused ultrasound system, cavitation produced by each insonation period is indicated by a separate indicator as in FIG. 7, but for each insonation period, as well as the location of the cavitation, the level of cavitation is indicated, for example as a greyscale value of the indicator circle 1000. The greyscale level may correspond to the total detected ultrasound energy detected from the cavitation during the insonation period, or the peak ultrasound power detected during the insonation period, or the proportion of the duration of the insonation period for which the detected ultrasound generated by the cavitation exceeded a threshold power. The overall effectiveness of the treatment may then by assessed on the basis of the number of focused ultrasound insonation periods that generate cavitation within the target volume 1002, the distribution of locations of cavitation within the target volume 1002, and the effectiveness of the cavitation from each of the insonation period, as indicted by the greyscale of each indicator 1000. This assessment may be made by the clinician on the basis of the visual feedback, or by the control unit on the basis of a simple algorithm combining the three parameters of the cavitation. Again the overall assessment by the control unit may result in the control unit generating a further visual indicator to the user on the display, either to provide information to the user or to provide directions to the user, or generating a control signal for example to the probe to stop the treatment.

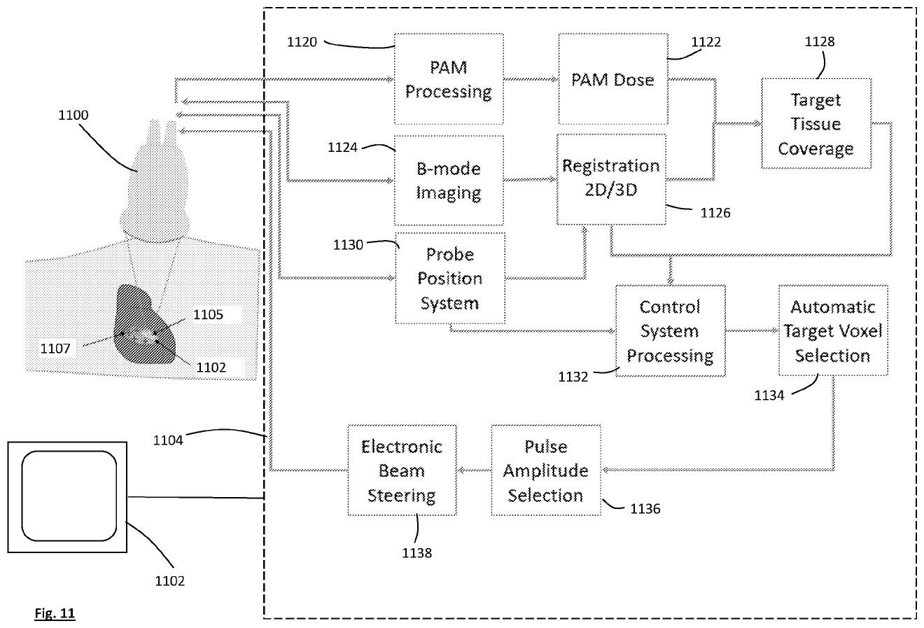

Referring to FIG. 11, in a system with a hand-held probe 1100, in which the GUI provides feedback to a skilled practitioner to assist them in performing the treatment, the processing system, typically implemented in software, includes a number of functional units as will now be described. The probe 1100, processing unit 1104, and the GUI 1110 are similar to those of FIG. 1. The functional units within the processing unit 1104 include, for example, a PAM processing unit 1120 arranged to perform the PAM processing to form an image of the cavitation, a PAM dose calculation unit 1122 arranged to calculate the treatment dose (or efficacy) at each position based on the imaged cavitation and other factors as described above, a B-mode imaging unit 1124 arranged to form B-mode ultrasound images of the patient, an image registration unit 1126 arrange to register the cavitation images, the B-mode images, and a 3D patient image, and a target tissue coverage determination unit 1128 arranged to determine the treatment coverage of a target region 1102 based on the dose from each treatment pulse. A probe positioning unit 1130 is arranged to determine the position of the probe 1100 and therefore also the position, or the range of possible positions, of the ultrasound focal region, and to locate those on the 3D image. That position, or range of positions, can be indicated on the 3D image to the clinician, for example as ‘cross hairs’ or a highlighted volume, showing where treatment is expected to occur if the focused ultrasound is triggered. A control system processing unit 1132 may be provided which is arranged to analyse data from the target tissue coverage unit 1128, the registration unit 1126 and the probe position system 1130 so as to identify parts 1105 of the target region 1102 which have been treated and parts of the target region which have not been treated. An automatic target voxel selection unit 1134 may then be arranged to identify, after each pulse, a voxel (or location 1107 which may be indicated by a cross or any other suitable indicator) from the 3D image for the next treatment pulse. A pulse amplitude selection unit 1136 may be included which is arranged to determine, for example based on the amplitude of previous pulses and images of cavitation from those previous pulses, an amplitude for the next treatment pulse. If the probe is arranged to steer the focused ultrasound beam electronically rather than just relying on manual control, a beam steering unit 1138 may be included to determine when the position of the target voxel, relative to the probe, is such that it is within a reachable field of view of the focused ultrasound beam, and steer the focused ultrasound beam to the target voxel or provide visual instruction on the display means to the practitioner on the position/angle of the probe relative to current position. The next focused ultrasound pulse may then be triggered automatically by the system when the target voxel is within range, or left to the control of the clinician.

In use, the 3D image of the patient is marked up with the target region 1102 in advance of treatment. This may include the target treatment locations 1107 which may be indicated on the image on the GUI for the clinician. During treatment the probe 1100 generates a B-mode image of the part of the patient within its field of view, as well as a PAM image of the cavitation as it is produced during each focused ultrasound pulse. The processing unit 1104 also generates on the 3D image the indicators 1107 showing the clinician where the focal region of the probe should be directed, as well as the cross hairs or other indication, of where the focal region of the probe is located in real time. The clinician can then move the probe prior to triggering each pulse or insonation period to align the cross hairs with a target position 1107 so that the FUS will produce treatment at a desired location within the target region 1102. After each pulse the target tissue coverage unit 1130 can determine the efficacy of the treatment at the target position and indicate that to the clinician, for example by changing the colour of the image at or around the target position after effective treatment is identified. This feedback enables the clinician to continue with the treatment until the whole, or a sufficient part, of the target region 1102 has been treated.

Referring to FIG. 12 in a fully automated robotic system, the probe 1200 is held on a robot arm 1201 and the processing unit 1204 is arranged to provide control inputs to an actuator unit 1205 to control the movement of the robot arm 1201. The functional units within the processing unit 1204 include, for example, a PAM processing unit 1220, a PAM dose calculation unit 1222, a B-mode imaging unit 1224, an image registration unit 1226, and a target tissue coverage determination unit 1128 which perform the same functions as in the embodiment of FIG. 11. A control system processing unit 1232 and an automatic target voxel selection unit 1234 may also be provided and arranged to identify, after each pulse, and based on the efficacy of previous pulses and the location of cavitation that they produced, a voxel (or location) from the 3D image (or a 3D image data set) for the next treatment pulse. An arm movement control and positioning unit 1238 may be included which is arranged to generate the control inputs to the actuator unit 1205, based on the position of the target voxel relative to the current position and orientation of the probe 1200. A pulse amplitude selection unit 1236 may be included. If the probe is arranged to steer the focused ultrasound beam electronically rather than just relying on control of the robot arm 1201, a beam steering unit may be included to add further control to the position of the focused ultrasound focus.

In operation, once the target region 1202 around the tumor 1203 has been identified in the 3D image of the patient, the processing unit 1204 may be arranged to identify a number of target positions 1207 (numbered 1, 2 . . . . N in FIG. 12) within the target region 1202, control the robot arm 1201, and the focused ultrasound beam steering if that is included, so as to move the focused ultrasound focus to one of the target positions, and then to trigger the next focused ultrasound pulse. During the pulse the cavitation is imaged by the PAM processing unit 1220 and the PAM dose for the resulting cavitation position determined by the PAM dose unit 1222. The dose may be measured after completion of the pulse and the effectiveness of the treatment determined by the target tissue coverage unit 1228. The target voxel selection for the next pulse may then be dependent on the efficacy of the previous pulse, and indeed all previous pulses. After each pulse the processing unit target tissue coverage unit 1228 may be arranged to determine whether the treatment is complete, for example by simply determining that all of the target positions 1207 have been treated, or by determining that all of the target positions have been treated effectively, based on an efficacy measure for each target position. Alternatively each of the target positions 1207 may be determined only after completion of the previous pulse, and the treatment continued until a predetermined number, or distribution, of positions within the target region 1202 are treated effectively.

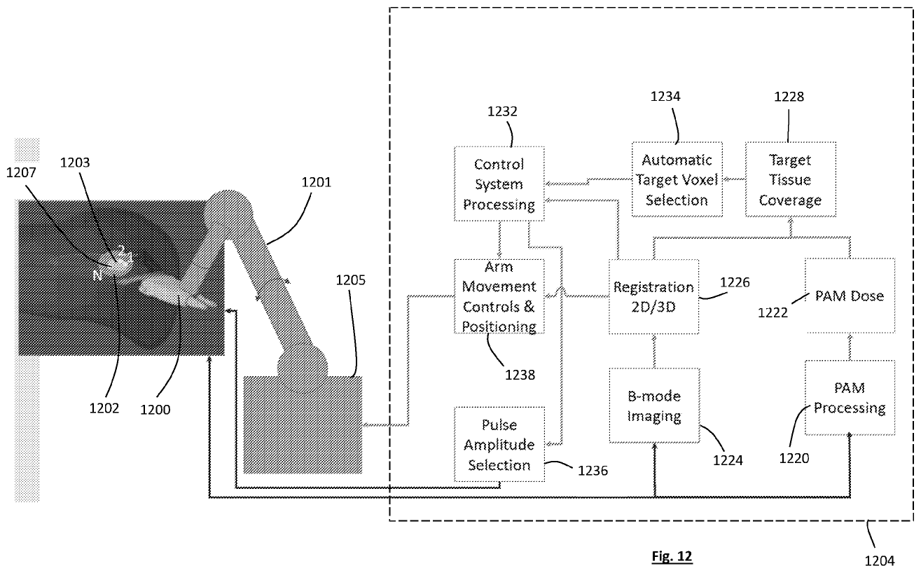

Referring to FIG. 13 the probe 1300 may be fixed relative to the patient, for example by being held against the patient's body, and the control and movement of the focused ultrasound focus controlled entirely by electronic beam steering. In this case the functional units of the processing unit 1304 are the same as for the hand-held probe, and indicated by the same reference numerals but increased by 200. The treatment operation is the same, except that the processing unit 1304 automatically progresses from each pulse to the next adjusting the focal position of the focused ultrasound beam until it determines that the treatment is complete.

In order to test the relationship between various PAM parameters and the efficacy of drug delivery, in an in-vivo experiment, mice were implanted with human breast cancer cells (4T1) and allowed to grow to approximately 100 mm\hat{ }3. The mice were then treated with either antibody alone (Cetixumab) or cavitation-assisted antibody delivery from SonoTran particles and SonoTran System ultrasound exposure for 10 min after bolus injection of the particles and antibody. In these tests because the tumors were small the FUS focal region was held stationary during insonation, resulting in a single fixed focal region. The mice in the treatment group had a statistically significant increase in cetuximab concentration in tumors measured by anti-human IgG ELISA. Paired t-test was used for the statistical analysis (*P<0.05). Various temporal and spatial metrics of cavitation (via PAM) were measured and used that to study at their correlation to delivery of the drug.

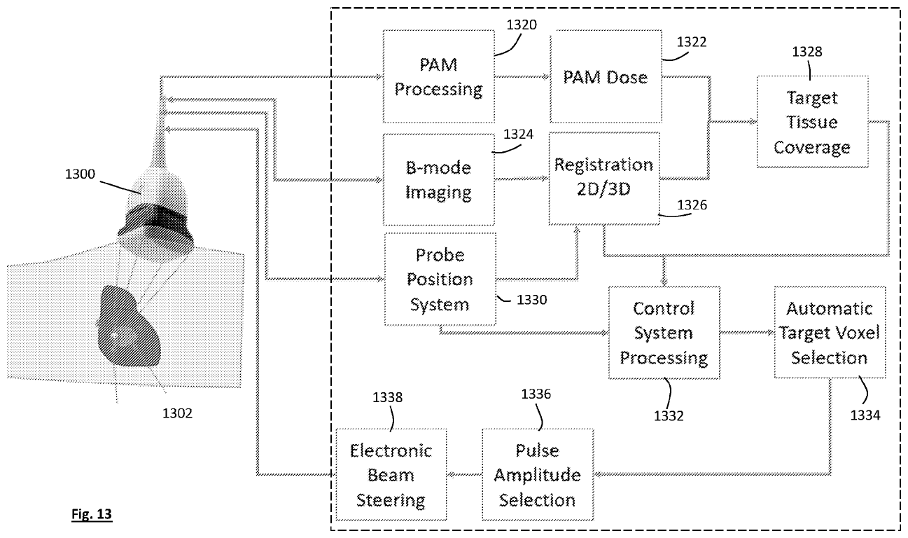

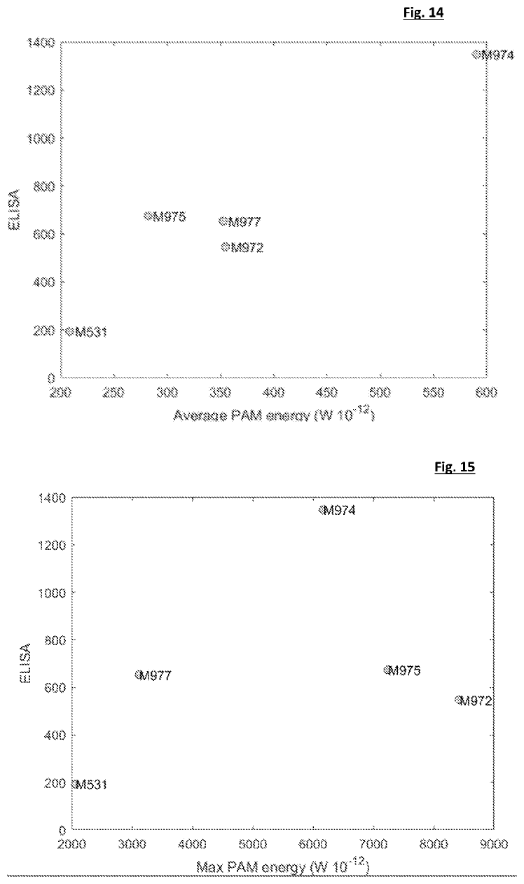

Referring to FIG. 14, if the ELISA measure is plotted against the average PAM energy detected, with each point representing the measurements for a respective mouse, this shows a good correlation between drug delivery and average PAM energy levels. Referring to FIG. 15, if the ELISA measure is plotted against maximum PAM energy there is some correlation but it is less strong than for the average PAM energy. Referring to FIG. 16 if the ELISA measure is plotted against maximum cumulative PAM energy over the 10 min insonation period, there is similar correlation to the average PAM energy measurements.

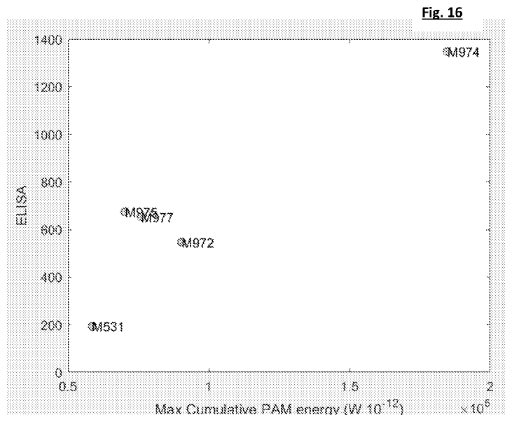

Referring to FIGS. 17, for mouse M531 which had the lowest ELISA delivery it can be seen that the cumulative dose, indicated by dots showing the locations of the focused ultrasound focal region for each pulse, is spread out. Referring to FIG. 18, for the same mouse it can be seen that the max PAM energy varies considerably over the course or treatment. In comparison, referring to FIG. 19 the cumulative does for mouse M974 which had the highest ELISA delivery, the cumulative dose is much more narrowly concentrated and the max PAM energy was maintained at significant levels for a larger proportion of the time. This shows, as expected, that sustained and concentrated cavitation is more effective than more spread cavitation at lower levels for this tumor model and this drug.

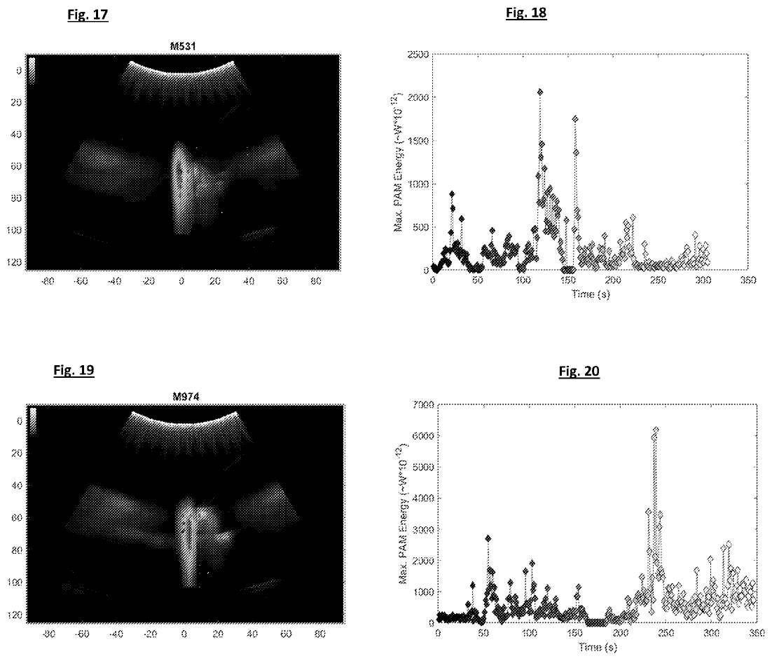

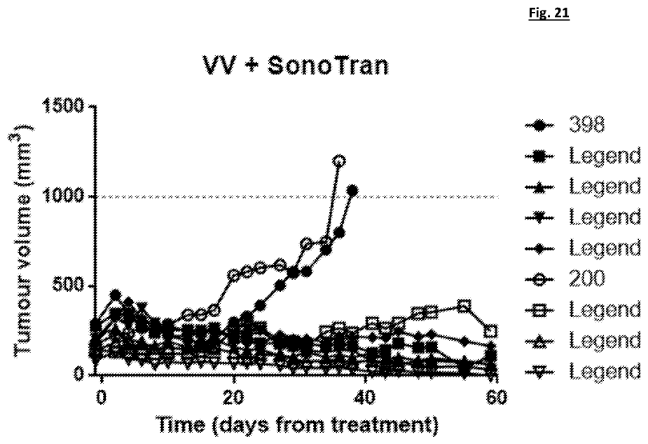

Referring to FIG. 21 in a further study using vaccinia virus to control the growth of tumors in mice, vaccinia virus and SonoTran particles were injected, and the mice then treated with the SonoTran System ultrasound exposure and the effect on growth of the tumours monitored. As can be seen, in most of the mice, growth was well controlled, but in two mice (M200 and M389) the growth was not well controlled.

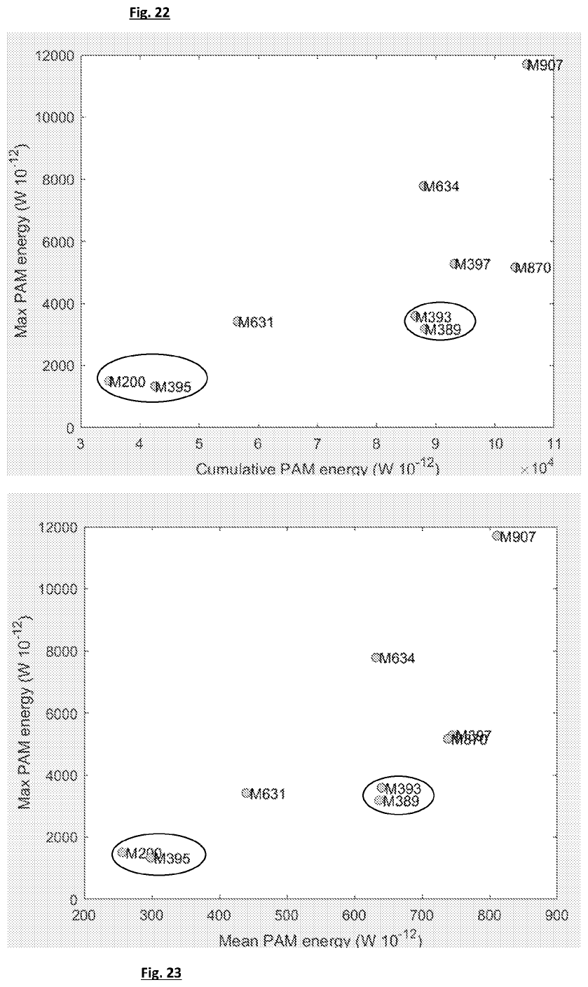

As shown in FIG. 22, if maximum PAM energy is plotted against Cumulative PAM energy for the different mice from FIG. 21, the two mice M200 and M389 cannot be distinguished from the others. Similarly referring to FIG. 23, plots of Mean PAM energy against max PAM energy also do not distinguish the two mice M200 and M389 from the others.

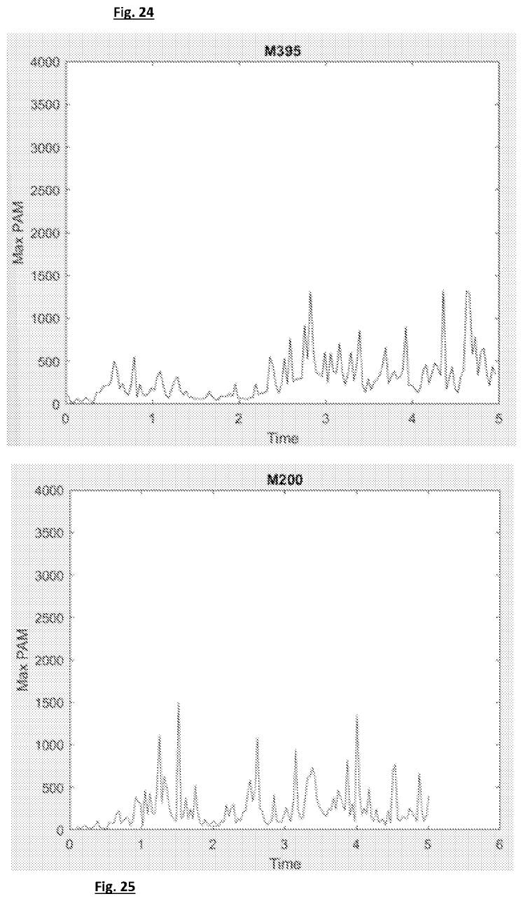

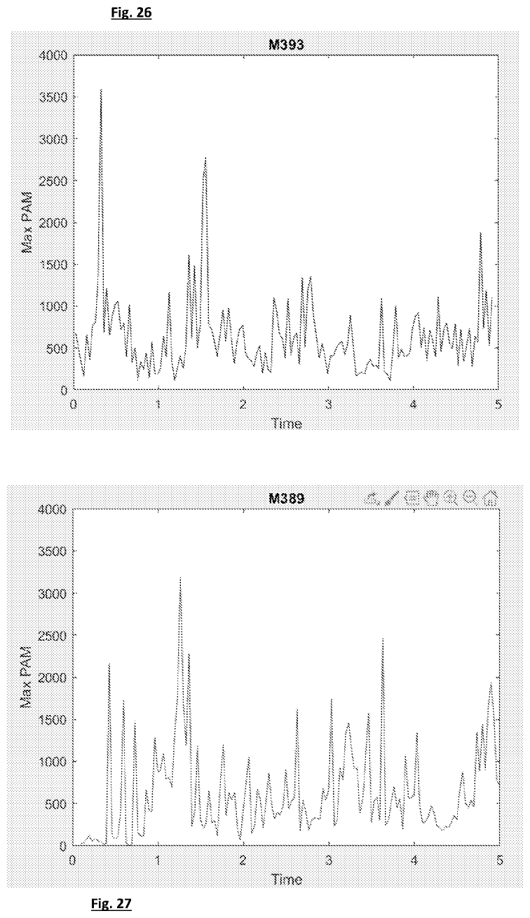

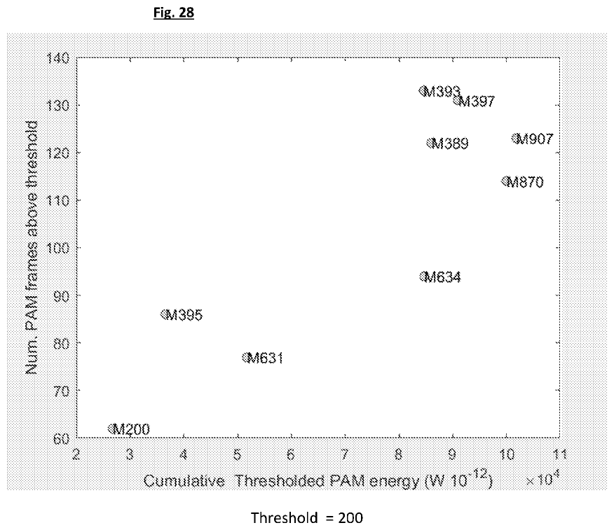

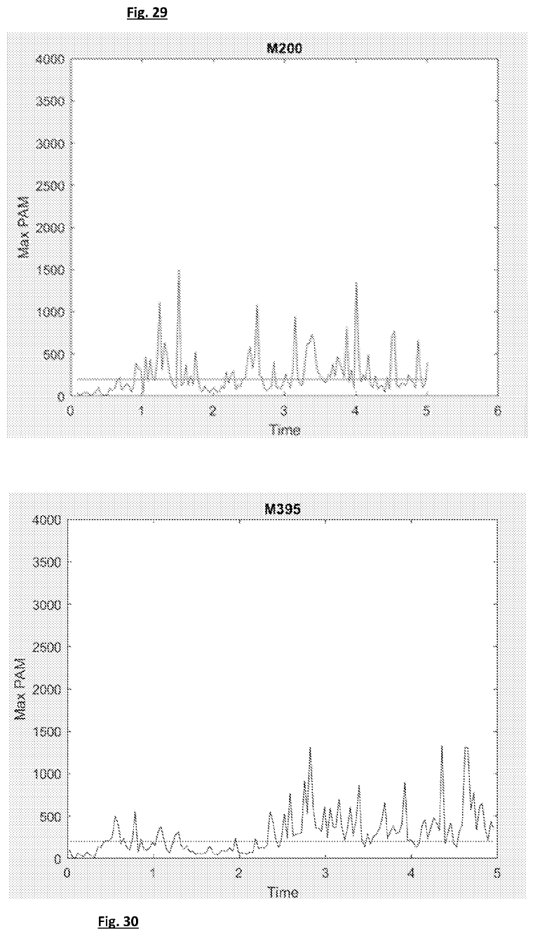

Referring to FIGS. 24 to 27 if max PAM energy is plotted against time for the four mice M395, M200, M393 and M389 it can be seen that there is a more consistently high max PAM for the two mice M395 and M393 compared to mouse M200. Referring to FIG. 28 if cumulative thresholded PAM energy is plotted against the number of frames during the treatment for which PAM energy was above a threshold, one of the mice M200 for which the tumour growth was not controlled can be distinguished from the others. This shows that thresholded cavitation levels can be a better indicator of efficacy of treatment than simple cavitation levels. FIGS. 29 and 30 show the threshold level on the plots of FIGS. 24 and 25.

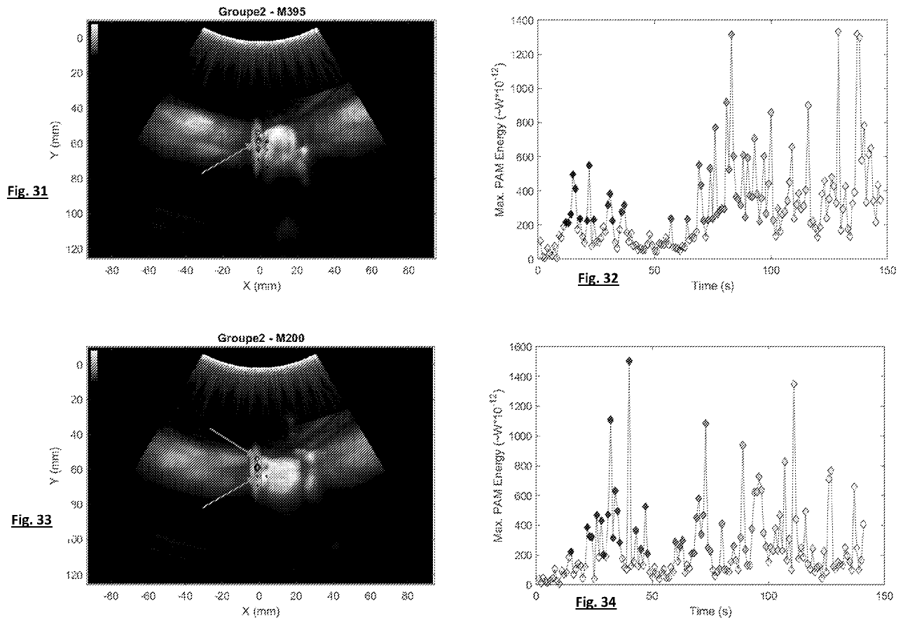

Referring to FIGS. 31 and 32, for mouse M395 the main areas of cavitation indicated by the arrow on FIG. 31 were within the tumor, and the cumulative cavitation was at a relatively high level as shown in FIG. 32. This compares with mouse M200 for which the main areas of cavitation were at the periphery of the tumor as highlighted in FIG. 33, as well as the cumulative cavitation being slightly lower as shown in FIG. 34. This accounts for the relatively poor control of tumor volume in M200. This shows, as would be expected, that the position of the cavitation within the tumor is important, and generating cavitation within the tumor is more effective than generating cavitation at the edge of the tumor.

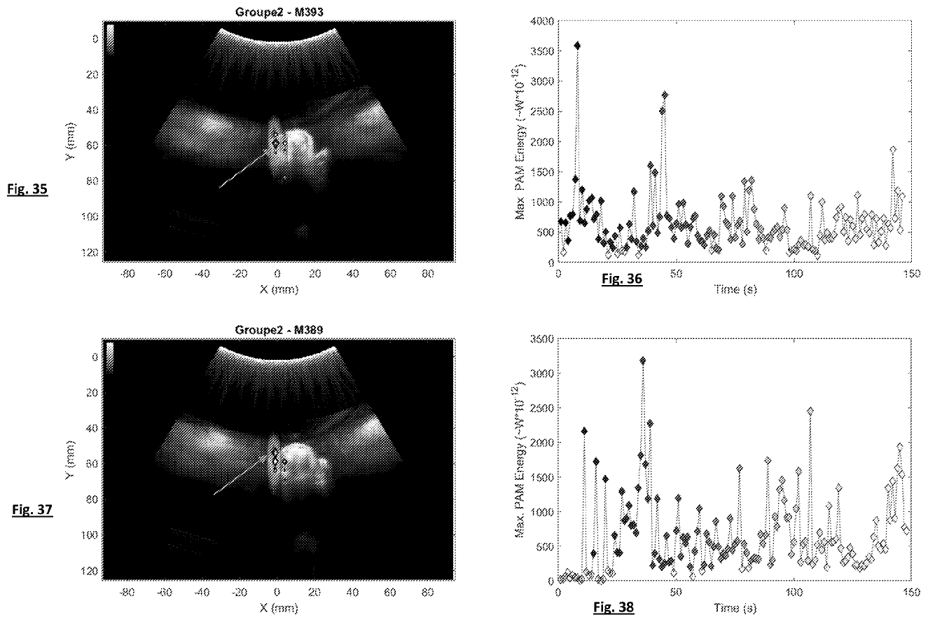

Referring to FIGS. 35 and 36, for mouse M393 again the largest cavitation events occur within the tumor, whereas referring to FIGS. 37 and 38, for mouse M389 the largest cavitation events are on the periphery of the tumor. This again shows that the different results for these two mice can be explained by the different positions of the cavitation within the respective tumors.

1-20. (canceled)

21. An apparatus for monitoring drug delivery to a subject, the apparatus comprising:
a transducer transmitting pressure waves towards the subject thereby generating cavitation at the subject;
an array of detectors detecting pressure waves generated by the cavitation;
a processor receiving signals generated by the detectors and processing the signals to determine a parameter representing a location of the cavitation and at least one further parameter of the cavitation; and
the processor generating from the at least one further parameter an efficacy indicator indicative of an efficacy of drug delivery at the location of the cavitation.

22. The apparatus according to claim 21 wherein the processor comprises a memory having stored therein a relationship between values of the at least one further parameter and values of the efficacy indicator, and wherein the processor generates the efficacy indicator based upon a value of the at least one further parameter and the stored relationship.

23. The apparatus according to claim 21 wherein the processor maps the location of the cavitation onto an image of the subject.

24. The apparatus according to claim 23 wherein the image of the subject includes a definition of a region for treatment.

25. The apparatus according to claim 21 wherein the transducer transmits the pressure waves towards a focal region within the subject and the processor moves the focal region whereby the cavitation is generated at a plurality of locations within the subject.

26. The apparatus according to claim 25 wherein the transducer transmits the pressure waves over a plurality of transmission periods and the processor moves the focal region between consecutive ones of the transmission periods.

27. The apparatus according to claim 21 wherein the transducer is manually movable to move the focal region within the subject whereby the cavitation is generated at a plurality of locations within the subject.

28. The apparatus according to claim 21 wherein the efficacy indicator comprises an efficacy image feature, and the processor controls a display to display an image of the subject together with the efficacy image feature.

29. The apparatus according to claim 28 wherein the efficacy image feature is located on the image of the subject at a position corresponding to the location of the cavitation in the subject.

30. The apparatus according to claim 29 wherein the processor generates a one of the efficacy indicator for each of a plurality of locations of the cavitation in the subject.

31. The apparatus according to claim 21 wherein the efficacy indicator comprises a control signal.

32. The apparatus according to claim 31 wherein the control signal is adapted to control a user interface.

33. The apparatus according to claim 31 wherein the efficacy indicator comprises an efficacy image feature and wherein the control signal is adapted to control a display to display an image of the subject together with the efficacy image feature.

34. The apparatus according to claim 31 wherein the control signal is adapted to control the transducer.

35. The apparatus according to claim 34 wherein the control signal is adapted to control the location of the cavitation by moving the transducer.

36. The apparatus according to claim 21 wherein the at least one further parameter of the cavitation is at least one of: detected ultrasound energy; mean detected ultrasound energy; maximum detected ultrasound energy; and thresholded detected ultrasound energy.

37. The apparatus according to claim 21 wherein the processor is adapted to identify a plurality of cavitation regions, and the at least one further parameter is a relative position of each of the cavitation regions.

38. The apparatus according to claim 37 wherein the processor is adapted to process signals received from the detectors over a plurality of detection periods and to identify a one of the cavitation regions associated with each of the detection periods.

39. The apparatus according to claim 21 including a user input adapted to input a tissue type to the processor, wherein the generated efficacy indicator is dependent on the tissue type.

40. The apparatus according to claim 21 including a user input adapted to input a drug class, wherein the generated efficacy indicator is dependent on the drug class.
